(19) United States
(12) Patent Application Publication
(10) Pub. No.: US 2026/0295072 A1
(43) Pub. Date: Oct. 1, 2026

(54) POLYMER NANOPARTICLE COMPOSITIONS FOR NON-VIRAL GENE DELIVERY

(71) Applicant: BATTELLE MEMORIAL INSTITUTE, Columbus, OH (US)

(72) Inventors: Andrea D. McCue, Columbus, OH (US); Ashlee J. Colbert, Blacklick, OH (US); Molly Kaufman, Columbus, OH (US); Danielle J. Huk, Columbus, OH (US); Kenneth R. Sims, Jr., Delaware, OH (US); Emma K. Schmitz, Columbus, OH (US); Dean Constantine, Columbus, OH (US)

(21) Appl. No.: 19/629,483

(22) Filed: Mar. 26, 2026

Related U.S. Application Data
(60) Provisional application No. 63/777,971, filed on Mar. 26, 2025.

(51) Int. Cl. A61K 47/69; A61K 47/60

(52) U.S. Cl. CPC A61K 47/6933; A61K 47/60

(57) ABSTRACT

The disclosure relates to block copolymer nanoparticles for in vivo screening and for in vivo therapeutic delivery, and methods therefor. More particularly, the invention relates to polymer nanoparticles, such as reversible addition-fragmentation chain transfer (RAFT) polymer compositions, for delivering circRNA.

### RELATED APPLICATIONS

This application claims the benefit of U.S. Provisional Application 63/777,971, filed Mar. 26, 2025, the entire disclosure of which is incorporated herein by reference.

### BACKGROUND

Genetic medicines (including gene therapy, gene silencing, splicing regulators, and nucleic acid-based gene editors) are poised to produce revolutionary treatments, including vaccines, infectious disease treatments, antimicrobial treatments, antiviral treatments, and most notably, genetic disease treatments. However, the in vivo delivery of these genetic medicine payloads to the specific tissues and cells that need to be treated, while avoiding tissues and cells that can reduce the efficacy or safety of the genetic medicine, poses a significant challenge. Adeno-associated viruses (AAVs) are the most widely used tool for genetic medicine delivery, but AAVs are limited in their payload size, and they sometimes trigger unwanted immune responses, including the generation of anti-AAV antibodies, a cell mediated response. Some of the immune responses caused by AAV in patients are potentially fatal immune responses.

Therapeutics based on the CRISPR/Cas9 system have an exceptional potential to treat a number of genetic diseases due to the capability of this system for precise and programmable gene editing. Gene editing and repair using the CRISPR/Cas9 system has two main mechanisms, including non-homologous end joining (NHEJ) which repairs the site of cut by inducing random indel mutation, and homology-directed repair (HDR), which repairs the cut site based on a pre-existing template. Because a pre-designed template can be used for HDR-directed repair, therapies based on this mechanism can be tailored to cure a large number of different genetic diseases. However, the main challenge is that HDR repair requires the delivery of CRISPR/Cas9, small guide RNA (sgRNA), and a donor DNA strand at the same time to a particular location. This requirement becomes particularly limiting for in vivo applications because ensuring co-delivery of multiple large molecules to the same targeted location is difficult. For example, the Cas9 enzyme sequence and guide RNA complex is too large to fit into AAVs.

Thus, there is a need for effective non-viral delivery systems, including gene delivery systems. The current state-of-the-art non-viral gene delivery systems, such as liposomes, have many drawbacks such as poor biocompatibility and the inability to easily engineer or functionalize them. Additional concerns are that such non-viral gene delivery systems are easily degraded by various enzymes as they pass through intracellular or intercellular compartments.

### SUMMARY

In certain aspects, the disclosure is directed to block copolymers. In certain aspects of the present disclosure, a block copolymer comprises
a first block comprising a monomer unit represented by formula I:

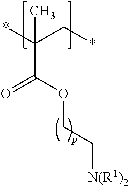

or a salt thereof, wherein:
   p is 0 or an integer selected from 1-3,
   each R¹ is individually selected from H or alkyl; and
   wherein each * represents a connecting point to the rest of the block copolymer; and
a second block comprising a copolymer of monomer units selected from formula II:

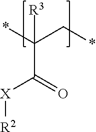

or a salt thereof, wherein:
   X is —O—;
   R² is alkyl; and
   R³ is alkyl;
   wherein each * represents a connecting point to the rest of the block copolymer; and
   formula III:

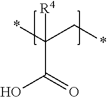

or a salt thereof, wherein:
   R⁴ is alkyl;
      wherein each * represents a connecting point to the rest of the block copolymer

In certain aspects, the disclosure relates to a polymer nanoparticle comprising the block copolymer according to the present disclosure.

In certain aspects, the disclosure relates to a composition comprising:
a polymer nanoparticle according to the present disclosure, and
a nucleic acid (e.g., a circular RNA) complexed to the polymer nanoparticle.

In certain aspects, the disclosure relates to a method of treating a disease comprising administering a therapeutically effective amount of a composition according to the present disclosure to a patient in need thereof.

In certain aspects, the disclosure relates to a method of transfecting a cell comprising contacting the cell with an effective amount of a composition according to the present disclosure.

Additional embodiments, features, and advantages of the disclosure will be apparent from the following detailed description and through practice of the disclosure.

### BRIEF DESCRIPTION OF THE DRAWINGS

FIG. 1 shows transfection efficiency from in vitro transfection of eGFP circRNA with PNP delivery vehicles into HEK293T cells.

FIG. 2 shows viability from in vitro transfection of eGFP circRNA with PNP delivery vehicles into HEK293T cells.

FIG. 3 shows cell counts from in vitro transfection of eGFP circRNA with PNP delivery vehicles into HEK293T cells.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended clauses.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” may be an approximation of ±10%, ±5%, or ±1%. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various [symbol] embodiments described herein, “*”, “**” and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by

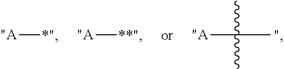

where each of “-*”, “-**”, and

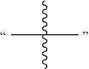

represents a bond to A and the point of covalent bond attachment to B. Alternatively, in some embodiments, the portion of A-B defined by the group or chemical structure B can be represented by

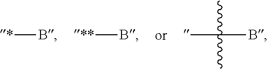

where each of “-*”, “**” and

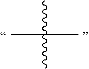

represents a bond to B and the point of covalent bond attachment to A.

As used herein, “molecular weight” refers to the weight average molecular weight (Mw) determined by a conventional polystyrene standard curve gel permeation chromatography method (hereinafter referred to as GPC) using multiple narrow distribution polystyrene standard samples relevant to the present invention and preferably between 2 kDa to 1,000 kDa.

As used herein, “degree of polymerization (DP)” refers to the number of monomer units in a polymer chain, such as in a block of a block copolymer. Methods of determining degree of polymerization (DP) are known in the art.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit disease may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a compound or a composition which reduces the frequency of, or delays the onset of, symptoms of a medical condition in a subject relative to a subject which does not receive the compound or composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance (e.g., a compound, a composition, or an agent) to a subject can be carried out using one of a variety of methods known to those skilled in the art. For example, a substance can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A substance (e.g., a compound, a composition, or agent) can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance (e.g., a compound, a composition, or an agent) to a subject will also depend, for example, on the age and/or the physical condition of the subject and the chemical and biological properties of the compound or agent (e.g., solubility, digestibility, bioavailability, stability and toxicity). In some embodiments, a substance (e.g., a compound, a composition, or an agent) is administered orally, e.g., to a subject by ingestion. In some embodiments, the orally administered substance is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated. The skilled worker can readily determine the effective amount for a given situation by routine experimentation.

### Definitions

The term “alkyl” refers to a straight- or branched-chain monovalent hydrocarbon group. The term “alkylene” refers to a straight- or branched-chain divalent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be unsubstituted or substituted as described herein. An alkyl or alkylene group can be substituted with any of the substituents in the various embodiments described herein, including one or more of such substituents.

The term “alkenyl” refers to a straight- or branched-chain mono-valent hydrocarbon group having one or more double bonds. In some embodiments, it can be advantageous to limit the number of atoms in an “alkenyl” to a specific range of atoms, such as C₂-C₂₀ alkenyl, C₂-C₁₂ alkenyl, or C₂-C₆ alkenyl. Examples of alkenyl groups include ethenyl (or vinyl), allyl, and but-3-en-1-yl. Included within this term are cis and trans isomers and mixtures thereof. It will be appreciated that an alkenyl can be unsubstituted or substituted as described herein. An alkenyl group can be substituted with any of the substituents in the various embodiments described herein, including one or more of such substituents.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀ alkyl indicates a hydrogen where the group is in a terminal position, a bond if internal. A C₁₋₆ alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

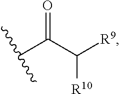

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

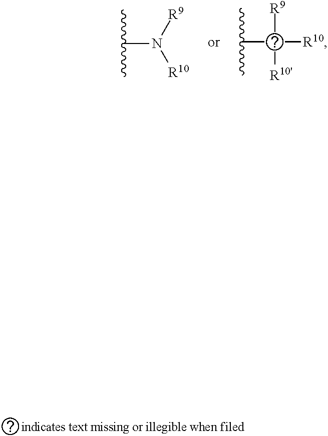

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “carboxy”, as used herein, refers to a group represented by the formula —CO₂H.

The term “ester”, as used herein, refers to a group —C(O)OR⁸ wherein R⁸ represents a hydrocarbyl group.

The term “ketone”, as used herein, refers to a group —C(O)R⁷ wherein R⁷ represents a hydrocarbyl group (e.g., alkyl, aryl, heteroaryl).

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group OSO₃H, or a pharmaceutically acceptable salt thereof.

The term “sulfoxide” is art-recognized and refers to the group S(O).

The term “sulfonate” is art-recognized and refers to the group SO₃H, or a pharmaceutically acceptable salt thereof.

The term “sulfone” is art-recognized and refers to the group S(O)₂.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

The term “substituted” refers to moieties having substituents replacing a hydrogen on one or more atoms (e.g., carbon atoms) of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The nomenclature “(ATOM)ᵢ.(ATOM)ⱼ” with j>i, when applied herein to a class of substituents, is meant to refer to embodiments of this disclosure for which each and every one of the number of atom members, from i to j including i and j, is independently realized. By way of example, the term C₁-C₃ refers independently to embodiments that have one carbon member (C₁), embodiments that have two carbon members (C₂), and embodiments that have three carbon members (C₃).

“Acceptable salt” or “salt” is used herein to refer to an acid addition salt or a basic addition salt which is suitable for or compatible with the compounds or a desired treatment.

The disclosure also includes acceptable salts (e.g., pharmaceutically acceptable salts) of the block copolymers described herein, preferably of those described above and of the specific compounds exemplified herein, and pharmaceutical compositions comprising such salts, and methods of using such salts.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. ^{⊖},” wherein “W^{⊖}” is an inorganic counter ion (e.g., an inorganic anion) or an organic counter ion (e.g., an organic anion). In certain embodiments, W^{⊖} is an anion that is complexed with a cation of a compound of the disclosure to form a pharmaceutically acceptable salt.

It will be understood that the chemical entities described herein, can exist as a salt of a free acid or base of a compound represented herein and an inorganic or organic counter ion. Illustratively, the salt can be formed during the manufacture of the compound (e.g., a salt or a pharmaceutically acceptable salt) or can substituted to a salt for further manufacture, formulation, or administration reasons. As illustrated herein, certain compounds include a “W^{⊖},” wherein “W ^{⊖}” is an inorganic counter ion (e.g., an inorganic anion) or an organic counter ion (e.g., an organic anion). In certain embodiments, W^{⊖} is an anion that is complexed with a cation of a compound of the disclosure to form a pharmaceutically acceptable salt.

The term “inorganic counter ion” represents an inorganic ion that accompanies an ionic species in order to maintain electric neutrality. An inorganic counter ion may represent an anion or cation. An inorganic counterion may accompany a free acid or base of a compound represented herein. An inorganic ion may form by a reaction of an inorganic base or inorganic acid and a compound described herein that possesses a sufficiently acidic group, a sufficiently basic group, both types of functional groups, or more than one of each type.

The term “organic counter ion” represents an organic counter ion that accompanies an ionic species in order to maintain electric neutrality. The organic ion may represent an anion or cation. An organic counter ion may accompany a free acid or base of a compound represented herein. An organic ion may form by a reaction of an organic base or organic acid and a compound described herein that possesses a sufficiently acidic group, a sufficiently basic group, both types of functional groups, or more than one of each type.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a block copolymer that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

As used herein and in connection with chemical structures depicting the various embodiments described herein, a polymer may be arranged in any linear or branched configuration.

For example, a polymer comprising one or more monomer units may be arranged as a homopolymer, a block copolymer, a statistical copolymer, a random copolymer, or an alternate copolymer of different monomers linked together in an alternating fashion. In one embodiment, the polymers described herein may include repeating blocks, such as in a block copolymer, of units selected from formula I and formula II.

The term “block copolymer” is known in the art, and a representative definition is that a block copolymer is a polymer comprising molecules in which there is a linear arrangement of blocks, for example a block A linearly connected to a block B, where each of the blocks (e.g., block A and block B) comprises units derived from a characteristic species or combination of species of monomer such that at least one difference exists between the species of monomer(s) of the different blocks (e.g., the monomer species or combination of species of block A is different than the monomer species or combination of species of block B. Illustratively, a block polymer may be represented by a formula including a “-b-” identifier between each block. Illustratively, a block polymer may include monomers that are copolymerized in a random or statistical manner (e.g., within a block), which may be represented by a formula including a “-co-” identifier between monomers. For example, a block polymer comprising a first block of a DMAEMA homopolymer (p(DMAEMA)) and a second block of a BMA and MAA copolymer (p(BMA-co-MAA)) may be represented by the formula: p(DMAEMA)-b-p(BMA-co-MAA).

The term “statistical copolymer” is known in the art, and a representative definition is that a statistical copolymer can be a copolymer composed of monomers that form a sequence based on a statistical rule (e.g., Markovian statistics).

The term “random copolymer” is known in the art, and a representative definition is that a random copolymer describes a copolymer where the probability of finding a given type monomer residue at a particular point in the chain is equal to the mole fraction of that monomer residue in the chain and is independent of the neighboring units in the chain.

The term “alternate copolymer” is known in the art, and a representative definition is that an alternate copolymer describes a copolymer of monomers sequentially linked in a uniform pattern.

“Nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

The term “polynucleotide,” as used herein, means a molecule including one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose. As used herein, a polyribonucleotide sequence that recites thymine (T) is understood to represent uracil (U).

“Polydeoxyribonucleotides,” “deoxyribonucleic acids,” and “DNA” mean macromolecules that include multiple deoxyribonucleotides that are polymerized via phosphodiester bonds. “Polyribonucleotides,” “ribonucleic acids,” and “RNA” mean macromolecules that include multiple ribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), deoxyuridine triphosphate (dUTP) and deoxythymidine triphosphate (dTTP) dNTPs, and may include detectable tags, such as protein tags, luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof).

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, mRNA, circRNA, cDNA, gDNA, ssDNA, dsDNA or any combination thereof.

The term “vector” or “construct” designates a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA. DNA and RNA can be synthesized naturally (e.g. by DNA replication or transcription of DNA or RNA, respectively). DNA and RNA can also be chemically synthesized. RNA can be post-transcriptionally modified. The terms “target mRNA” and “target transcript,” “target sequence” are synonymous as used herein.

As used herein, “target sequence” refers to a contiguous portion of the nucleotide sequence of a nucleic acid molecule, which may be formed during the transcription of a gene, including mRNA that is a product of RNA processing of a primary transcription product.

“Targeting” an oligomeric compound to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent.

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNAi, shRNAi, endogenous microRNA, and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

The term “RNAi” and “RNA interfering” with respect to an agent of the invention, are used interchangeably herein. RNAi molecules are typically comprised of a sequence of nucleic acids or nucleic acid analogs, specific for a target gene. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA).

As used herein, “shRNA” or “small hairpin RNA” (also called stem loop) is a type of RNAi. shRNAs may be composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. shRNAs functions as RNAi but are further defined in that shRNA species are double stranded hairpin-like structure for increased stability. These shRNAs, as well as other such agents described herein, can be contained in plasmids, retroviruses, and lentiviruses and expressed from, for example, the pol III U6 promoter, or another promoter.

### REPRESENTATIVE EMBODIMENTS

This disclosure describes compositions of polymers (e.g., diblock copolymers) which self-assemble in aqueous conditions to form polymer nanoparticles (PNPs). In certain embodiments, the PNPs can complex nucleic acids and deliver the complexed nucleic acids to a desired location. In certain embodiments, the polymer can be combined with a nucleic acid payload (e.g., a circular nucleic acid, such as a circular RNA), to facilitate effective delivery of a high dose of payload but with a lower dose of PNP delivery vehicle (sometimes called dose sparing). This approach may allow for effective delivery of the target payload using less delivery vehicle, which may decrease effects that can arise from a high dose of delivery vehicle.

One of the many nucleic acid-based payloads that PNPs can deliver is circular RNA. A circular RNA was used as the nucleic acid payload to assess dose-dependent delivery of nucleic acids with PNPs in the examples disclosed herein. As a therapeutic, circRNA is an appealing system, as it has been reported to have higher a higher in vitro efficacy than engineered mRNA, and unlike mRNA, it does not require base modification for its stability. The circular nature of the molecules themselves also make the molecules more naturally stable, as they are not susceptible to degradation by exonucleases. Lastly, this payload bypasses the risk of genomic integration inherent in AAV delivery vehicles or DNA-based payloads.

In certain embodiments, the PNPs described herein may allow for in vitro delivery of eGFP circRNA by a p(DMAEMA)-b-p(BMA-co-PAA) PNP into HEK293-T cells. By investigating many dose permutations of PNP concentration and payload amount, a dose for which a high amount of payload is delivered with a lower amount of PNP can be identified, as suggested by the transfection efficiency and cell viability examples disclosed herein.

In certain embodiments, a block copolymer comprises a first block and a second block. The first block may comprise a homopolymer of poly(2-dimethylaminoethyl methacrylate), p(DMAEMA). The second block may comprise copolymer, such as a statistical copolymer (e.g., a random copolymer). For example, the second block may comprise a copolymer of an alkyl acrylate (e.g., methylmethacrylate or butylmethacrylate) and an acrylic acid (e.g., methacrylic acid (MAA), ethylacrylic acid (EAA), or propylacrylic acid (PAA)).

In certain embodiments, a block copolymer includes a first block having a monomer unit represented by formula I:

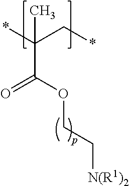

or a salt thereof, wherein:
p is 0 or an integer selected from 1-3,
each R¹ is individually selected from H or alkyl; and

wherein each * represents a connecting point to the rest of the block copolymer.

In certain embodiments, p is 0. In certain embodiments, p is 1, 2 or 3, and is preferably 1.

In certain embodiments, each R¹ is individually selected from H or alkyl (e.g., C₁-C₃ alkyl).

In certain embodiments, each R¹ is alkyl (e.g., C₁-C₃ alkyl) and each alkyl (e.g., C₁-C₃ alkyl) may be the same. In certain preferred embodiments, each R¹ is methyl.

In certain embodiments, the first block is a homopolymer comprising a plurality monomers of formula I. In certain embodiments, the first block comprises a plurality monomers of formula I, such as

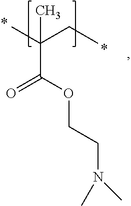

the corresponding polymer of which may be called poly(2-dimethylaminoethyl methacrylate), p(DMAEMA). Illustratively, the monomer unit represented by formula I is derivable from the polymerization of DMAEMA

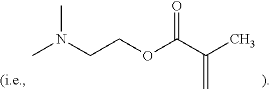

In certain embodiments, a block copolymer comprises a second block (also referred to as a copolymer second block) comprising a copolymer. The second block may comprise a copolymer formed of two or more monomer units, each individually represented by formula II:

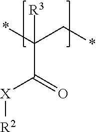

or a salt thereof, wherein:
X is —O—;
R² is alkyl; and
R³ is alkyl;
wherein each * represents a connecting point to the rest of the block copolymer; and
formula III:

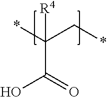

or a salt thereof, wherein:
R⁴ is alkyl;
wherein each * represents a connecting point to the rest of the block copolymer. For example, the copolymer can be a statistical copolymer (e.g., a random copolymer).

In certain embodiments, each of R², R³, and R⁴ is independently alkyl, such as C₁-C₆ alkyl (e.g., methyl, ethyl, propyl, or butyl).

In certain embodiments, the second block is a copolymer comprising (or consisting of) a first monomer of formula II and a second monomer of formula III.

In certain embodiments, R² is alkyl, such as C₁-C₆ alkyl (e.g., methyl, ethyl, propyl, or butyl), and is preferably butyl. For example, the monomer represented by formula II may be:

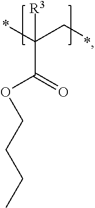

or a salt thereof.

In certain embodiments, R³ is alkyl, such as C₁-C₆ alkyl, and is preferably methyl. For example, the monomer unit represented by formula II may be:

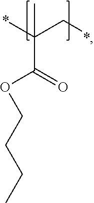

or a salt thereof, which may be referred to as butylmethacrylate (BMA), when present in the block copolymer. In certain preferred embodiments, the monomer unit represented by formula II is BMA. Illustratively, the monomer unit represented by formula II is derivable from the polymerization of BMA

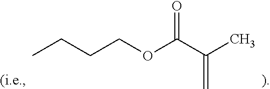

In certain embodiments, R⁴ is alkyl such as C₁-C₆ alkyl (e.g., methyl, ethyl, or propyl), and is preferably methyl, ethyl, or propyl. For example, the monomer unit represented by formula III may be:

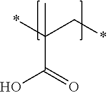

or a salt thereof, which may be referred to as methacrylic acid (MAA), when present in the block copolymer. Or, for example, the monomer unit represented by formula III may be:

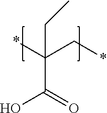

or a salt thereof, which may be referred to as ethylacrylic acid (EAA), when present in the block copolymer. Or, for example, preferably the monomer unit represented by formula III may be:

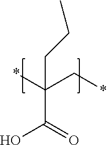

or a salt thereof, which may be referred to as propylacrylic acid (PAA), when present in the block copolymer. In certain preferred embodiments, the monomer unit represented by formula III is PAA. Illustratively, the monomer unit represented by formula III is derivable from the polymerization of

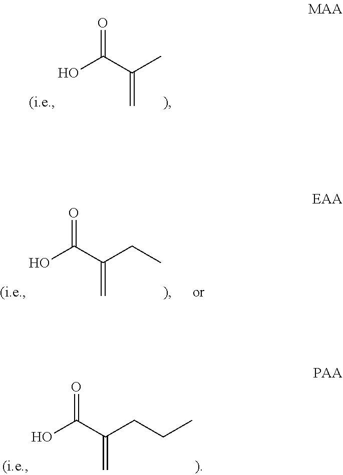

In certain embodiments, a block copolymer comprises a first block and a second block, the first block may comprise a homopolymer of poly(2-dimethylaminoethyl methacrylate) (p(DMAEMA)), and the second block may comprise a copolymer of an alkylacrylate (e.g., butylmethacrylate (BMA)) and an acrylic acid (e.g., methacrylic acid (MAA), ethylacrylic acid (EAA), or preferably propylacrylic acid (PAA)). In certain preferred embodiments, a block copolymer comprises a first block comprising a homopolymer of poly(2-dimethylaminoethyl methacrylate) (p(DMAEMA)), and a second block comprising a copolymer comprising (or consisting of) of butyl methacrylate (BMA) and propylacrylic acid (PAA).

In certain embodiments, a block copolymer is selected from p(DMAEMA)-b-p(BMA-co-MAA), p(DMAEMA)-b-p(BMA-co-EAA), and p(DMAEMA)-b-p(BMA-co-PAA), preferably p(DMAEMA)-b-p(BMA-co-PAA).

In certain embodiments, the copolymer has a total molecular weight of less than about 70 kDa. In some embodiments, the copolymer can have a total molecular weight of about 10 kDa to about 65 kDa, such as about 10 kDa to about 60 kDa (e.g., about 13 kDa to about 50 kDa). In certain embodiments, the copolymer has a total molecular weight of about 20 kDa to about 65 kDa, such as about 25 kDa to about 60 kDa. For example, the copolymer can have a total molecular weight of about 20 kDa to about 70 kDa, about 20 kDa to about 60 kDa, about 25 kDa to about 60 kDa, about 30 kDa to about 60 kDa, or about 40 kDa to about 50 kDa. In some embodiments, the copolymer can have a total molecular weight of about 15 kDa to about 50 kDa, about 15 kDa to about 40 kDa, about 15 kDa to about 35 kDa or about 25 kDa to about 35 kDa.

In certain embodiments, the first block has a molecular weight of about 10,000 Da to about 55,000 Da. In certain embodiments, the first block has a molecular weight of about 15,000 Da to about 55,000 Da. For example, the first block may be about 20,000 Da to about 55,000 Da, about 25,000 Da to about 55,000 Da, about 30,000 Da to about 55,000 Da, about 35,000 Da to about 55,000 Da, about 40,000 Da to about 55,000 Da, or about 45,000 Da to about 55,000 Da. In some embodiments, the first block may be about 15,000 Da to about 50,000 Da, about 15,000 Da to about 40,000 Da, or about 15,000 Da to about 25,000 Da. In certain embodiments, the first block has a molecular weight of about 15,000 Da to about 25,000 Da, such as about 18,000 Da to about 20,000 Da. In certain embodiments, the first block has a molecular weight of about 30,000 Da to about 40,000 Da, such as about 35,000 Da to about 38,000 Da. In certain embodiments, the first block has a molecular weight of about 40,000 Da to about 50,000 Da, such as about 44,000 Da to about 48,000 Da. In certain embodiments, the first block has a molecular weight of about 18,000 Da to about 48,000 Da.

In some embodiments, the first block has a degree of polymerization (DP), for example a theoretical DP. In certain embodiments, the first block has a theoretical degree of polymerization of about 250 to about 600, such as about 290 to about 600. For example, the first block may have a degree of polymerization of about 250 to about 500, about 250 to about 400, about 300 to about 600, about 300 to about 500, about 300 to about 400, about 400 to about 600, about 400 to about 500, or about 500 to about 600. In certain embodiments, the first block has a theoretical degree of polymerization of about 360, about 480, or about 600. In certain embodiments, the first block has a theoretical degree of polymerization of about 350 to about 400 (e.g., about 360). In certain embodiments, the first block has a theoretical degree of polymerization of about 450 to about 500 (e.g., about 480). In certain embodiments, the first block has a theoretical degree of polymerization of about 550 to about 600 (e.g., about 600).

In certain embodiments, the first block has a molecular weight of about 10,000 Da to about 55,000 Da, and a theoretical degree of polymerization of about 250 to about 600. In certain embodiments, the first block has a molecular weight of about 15,000 Da to about 25,000 Da, and a theoretical degree of polymerization of about 350 to about 400. In certain embodiments, the first block has a molecular weight of about 30,000 Da to about 40,000 Da, and a theoretical degree of polymerization of about 450 to about 500. In certain embodiments, the first block has a molecular weight of about 44,000 Da to about 48,000 Da, and a theoretical degree of polymerization of about 550 to about 600. In certain embodiments, the first block has a molecular weight of about 46,000 Da to about 50,000 Da, and a theoretical degree of polymerization of about 250 to about 300.

In certain embodiments, the second block has a molecular weight of about 1,000 Da to about 50,000 Da. For example, the second block may be about 1,000 Da to about 40,000 Da, about 1,000 Da to about 30,000 Da, about 1,000 Da to about 25,000 Da, about 1,000 Da to about 20,000 Da, about 1,000 Da to about 15,000 Da, about 1,000 Da to about 12,000 Da, about 1,000 Da to about 10,000 Da, about 1,000 Da to about 7,000 Da, or about 1,000 Da to about 5,000 Da. In certain embodiments, the second block has a molecular weight of about 1,000 Da to about 25,000 Da, such as about 1,000 Da to about 20,000 Da. In certain embodiments, the second block has a molecular weight of about 1,000 Da to about 10,000 Da. In certain embodiments, the second block has a molecular weight of about 25,000 Da to about 50,000 Da.

In certain embodiments, the second block has a degree of polymerization (DP), for example a theoretical DP, less than the first block. In certain embodiments, the second block has a theoretical degree of polymerization of about 15 to about 100, such as about 50 to about 85. In certain embodiments, the second block has a theoretical degree of polymerization of about 15 to about 175. For example, the second block may have a degree of polymerization of about 20 to about 150, about 20 to about 100, about 20 to about 75, about 25 to about 75, or about 25 to about 65. For example, the second block may have a theoretical degree of polymerization of about 20 to about 100, about 30 to about 100, about 40 to about 100, about 40 to about 90, or about 40 to about 80. In certain embodiments, the second block has a theoretical degree of polymerization of about 40 to about 80 (e.g., about 50). In certain embodiments, the second block has a theoretical degree of polymerization of about 45 to about 55. In certain embodiments, the second block has a theoretical degree of polymerization of about 55 to about 65. In certain embodiments, the second block has a theoretical degree of polymerization of about 65 to about 75. In certain embodiments, the second block has a theoretical degree of polymerization of about 75 to about 85. In certain embodiments, the second block has a theoretical degree of polymerization of about 50, about 60, about 70, or about 78.

In certain embodiments, the second block has a molecular weight of about 1,000 Da to about 50,000 Da, and a theoretical degree of polymerization of about 40 to about 100. In certain embodiments, the second block has a molecular weight of about 1,000 Da to about 25,000 Da, and a theoretical degree of polymerization of about 40 to about 100. In certain embodiments, the second block has a molecular weight of about 25,000 Da to about 50,000 Da, and a theoretical degree of polymerization of about 40 to about 100.

In certain embodiments, the block copolymer has an overall degree of polymerization, which is defined as the calculated from the sum of the degrees of polymerization of the individual blocks. In certain embodiments, the block copolymer has an overall degree of polymerization of about 300 to about 700. For example, the block copolymer may have an overall degree of polymerization of about 300 to about 600, about 300 to about 500, about 300 to about 400, about 400 to about 700, about 400 to about 600, about 400 to about 500, about 500 to about 700, or about 500 to about 600.

In certain embodiments, the second block of the copolymer has the monomers according to formula II (e.g., an alkyl methacrylate, such as BMA) and formula III (e.g., an alkyl acrylic acid, such as MAA, EAA, or PAA) present at a particular molar ratio of the repeating units. In some embodiments, the molar ratio of the compound of formula II (e.g., an alkyl methacrylate, such as BMA) and the compound of formula III (e.g., an alkyl acrylic acid, such as MAA, EAA, or PAA) in the second block is about 25:75 to about 75:25. In some embodiments, the molar ratio of the repeating units of the compound of formula II (e.g., an alkyl methacrylate, such as BMA) and the compound of formula III (e.g., an alkyl acrylic acid, such as MAA, EAA, or PAA) in the second block is about 30:70, about 40:60, about 50:50, about 60:40, or about 70:30. In some embodiments, the molar ratio of the compound of formula II (e.g., an alkyl methacrylate, such as BMA) and the compound of formula III (e.g., an alkyl acrylic acid, such as MAA, EAA, or PAA) in the second block is about 45:55, about 50:50, or about 55:45.

In certain embodiments, the first block has a molecular weight of about 10,000 Da to about 55,000 Da, and the second block has a molar ratio of the compound of formula II (e.g., an alkyl methacrylate, such as BMA) and the compound of formula III (e.g., an alkyl acrylic acid, such as MAA, EAA, or PAA) of about 25:75 to about 75:25. In certain embodiments, the first block has a molecular weight of about 15,000 Da to about 25,000 Da, and the second block has a molar ratio of the compound of formula II and the compound of formula of about 30:70 to about 70:30. In certain embodiments, the first block has a molecular weight of about 30,000 Da to about 40,000 Da, and the second block has a molar ratio of the compound of formula II and the compound of formula of about 40:60 to about 60:40. In certain embodiments, the first block has a molecular weight of about 44,000 Da to about 48,000 Da, and the second block has a molar ratio of the compound of formula II and the compound of formula of about 40:60 to about 60:40.

In some embodiments, block copolymers as prepared herein can be described by the following structure:

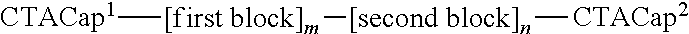

where each CTACap (e.g., CTACap¹ and CTACap²) is a capping unit derived from the chain transfer agent(s) used in the process for preparing the RAFT copolymer. The CTA used for preparing each of the first block and the second block can be the same or different. In some embodiments, the CTA used to prepare each the first block and the second block is the same (e.g., macroCTA). In some embodiments, the CTA used to prepare each of the first block and the second block is different. In some embodiments, the CTA used to prepare one or both of the first block and the second block comprises a functional group for the covalent attachment of a biomolecule, drug, or label to the block copolymer. In some embodiments, the covalent attachment can be via an ester or an amide bond. In some embodiments, the covalent attachment can be via EDC-NHS chemistry.

In some embodiments, the first block comprises a cap of formula:

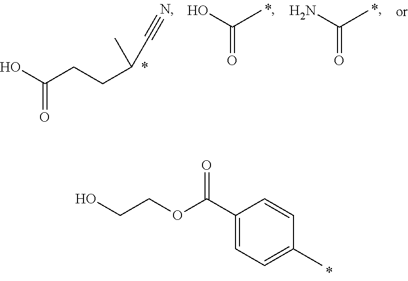

or a salt thereof, wherein * represents a point of covalent attachment to the first block. In certain preferred embodiments, the cap is

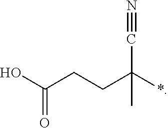

In some embodiments, the second capping unit is of formula

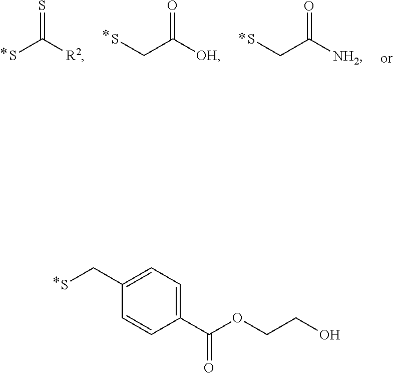

or a salt thereof, wherein * represents a point of covalent attachment to the second block, and R² is —SC₂-C₁₂ alkyl or C₆H₅, and is preferably

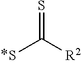

such as

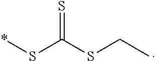 [SMILES: *SC(=S)SCC]

In some embodiments, a polymer nanoparticle comprises a block copolymer according to the present disclosure. In some embodiments, the block copolymer self-assembles into the nanoparticle.

In some embodiments, a composition comprises a polymer nanoparticle as described herein and a nucleic acid (sometimes called a payload) complexed to the polymer nanoparticle. For example, the nucleic acid may be complexed to the nanoparticle through electrostatic interactions. In certain preferred embodiments, the polymer nanoparticle of the composition serves as a transfection agent to deliver a nucleic acid to a cell.

In certain embodiments, the nucleic acid is an RNA (e.g., a circular RNA, an mRNA, a microRNA, antisense oligonucleotides, or an siRNA) or a DNA (e.g., an ssDNA, a dsDNA, or a complimentary coding DNA (cDNA), or a DNA plasmid (pDNA)). In certain embodiments, the nucleic acid is an mRNA. In certain preferred embodiments, the nucleic acid is a circular RNA. In certain embodiments, the nucleic acid is a DNA plasmid (pDNA).

The polymer nanoparticles described herein are capable of interacting with (e.g., encapsulating or complexing with) nucleotide plasmids. In some embodiments, the encapsulation efficiency is greater than about 70%, greater than about 75%, greater than about 80%, greater than about 85%, greater than about 90%, greater than about 95%, or greater than about 97%. In certain embodiments, the encapsulation efficiency is about 80% to about 95%.

The nanoparticles described herein are capable of encapsulating nucleic acids. In some embodiments, the nucleic acid is from about 10 bp to about 10,000 bp. In some embodiments, the nucleic acid is less than about 3,000 bp or less than about 2,500 bp. In some embodiments, the nucleic acid is about 500 bp to about 2,500 bp, about 500 bp to about 2,000 bp or about 1,000 bp to about 2,000 bp.

In certain embodiments, the ratio of nanoparticle to nucleic acid (e.g., circular RNA) is about 1:1 to about 30:1 by weight. For example, the ratio of nanoparticle to nucleic acid may be about 5:1 to about 30:1, about 7:1 to about 30:1, about 10:1 to about 30:1, about 15:1 to about 30:1, or about 20:1 to about 30:1 by weight. In some embodiments, the ratio of nanoparticle to nucleic acid is less than about 30:1, less than about 25:1, less than about 20:1, less than about 15:1, less than about 10:1, or less than about 7:1 by weight. In some embodiments, the ratio of nanoparticle to nucleic acid is about 1:1 to about 20:1 by weight. In some embodiments, the ratio of nanoparticle to nucleic acid is about 1:1 to about 10:1 by weight.

In certain embodiments, the ratio of nanoparticle to nucleic acid (e.g., circular RNA) is about 1:1 to about 25:1 by weight. For example, the ratio of nanoparticle to nucleic acid may be about 1:1 to about 20:1, about 1:1 to about 15:1, about 1:1 to about 10:1, about 1:1 to about 7:1, or about 1:1 to about 5:1 by weight. In some embodiments, the ratio of nanoparticle to nucleic acid is less than about 25:1, less than about 20:1, less than about 15:1, less than about 10:1, or less than about 7:1 by weight. In some embodiments, the ratio of nanoparticle to nucleic acid is about 1:1, about 2:1, about 3:1, about 4:1, about 5:1, about 6:1, about 7:1, about 10:1, about 15:1, about 20:1, or about 25:1 by weight. In some embodiments, the ratio of nanoparticle to nucleic acid is about 2.5:1 or about 5:1.

In certain embodiments, the payload (e.g., a nucleic acid, such as a circular RNA complexed to the polymer nanoparticle) may be present in the composition in an amount of about 150 ng to about 500 ng, such as about 150 ng to about 400 ng, about 200 ng to about 400 ng, or about 250 ng to about 400 ng. In certain embodiments, the payload may be present in the composition in an amount of at least about 150 ng, at least about 200 ng, at least about 250 ng, or at least about 300 ng.

In certain embodiments, the polymer nanoparticle may be present in the composition in an amount of about 0.001 mg/mL to about 0.05 mg/mL, such as about 0.001 mg/mL to about 0.03 mg/mL. In certain embodiments, the polymer nanoparticle may be present in the composition in an amount of less than about 0.05 mg/mL, less than about 0.04 mg/mL, or less than about 0.03 mg/mL.

In certain embodiments, the transfection efficiency is at least about 30%, at least about 50%, or at least about 70% into cells, for example in mammalian cells. In certain embodiments, the transfection efficiency is about 50% to about 99%, about 60% to about 99%, about 70% to about 99%, about 80% to about 99%, about 85% to about 99%, about 90% to about 99%, or about 95% to about 99%. In certain embodiments, the transfection efficiency is at least about 90% into cells, such as at least about 95% into cells.

In certain embodiments, the transfection efficiency is about 85% to about 99% and the ratio of nanoparticle to nucleic acid (e.g., circular RNA) is about 1:1 to about 30:1 by weight. For example, the transfection efficiency may be about 85% to about 99% and the ratio of nanoparticle to nucleic acid may be about 5:1 to about 30:1, about 7:1 to about 30:1, about 10:1 to about 30:1, about 15:1 to about 30:1, or about 20:1 to about 30:1 by weight. In certain embodiments, the transfection efficiency is about 85% to about 99% and the ratio of nanoparticle to nucleic acid is about 1:1, about 2:1, about 3:1, about 4:1, about 5:1, about 6:1, about 7:1, about 10:1, about 15:1, or about 20:1 by weight. In certain embodiments, the transfection efficiency is about 85% to about 99% and the ratio of nanoparticle to nucleic acid is about 1:1 to about 20:1. In certain embodiments, the transfection efficiency is about 85% to about 99% and the ratio of nanoparticle to nucleic acid is about 1:1 to about 10:1. In certain embodiments, the transfection efficiency is about 85% to about 99% and the ratio of nanoparticle to nucleic acid is about 1:1 to about 5:1.

In some embodiments, the polymer nanoparticles have low toxicity to cells. For example, in some embodiments, the cell viability after transfection of cells is at least about 90%, at least about 95%, or at least about 98%. In certain preferred embodiments, the cell viability after transfection of cells is at least 98%.

In certain embodiments, the nanoparticle comprises a barcode construct covalently attached to the nanoparticle, as described in U.S. Patent Application Publication No. 2022/033309, the entirety of which is hereby incorporated by reference. As described in U.S. Patent Application Publication No. 2022/033309, the presence of the barcode construct can allow for identification of PNPs of interest, for example by determining the presence of a PNP in particular tissue or by performance in an assay. It should be understood that although certain examples in this application are performed on PNPs that include the barcode, similar performance would be expected by PNPs that lack the barcode.

In some embodiments, a method of transfecting a cell is provided comprising contacting the cell with an effective amount of a composition according to the present disclosure. In some embodiments, the method is in vitro or in vivo.

The compositions described herein can be used for treating disease. For example, the polymer nanoparticles may be able to deliver a nucleic acid (i.e., a payload) that provides a therapeutic benefit to a patient. In certain embodiments, the polymer nanoparticle is able to deliver the nucleic acid to cells, such as neuronal cells. The compositions described herein can be used for treating disease, including neurological diseases (e.g., Parkinson's Disease or Alzheimer's Disease). In certain embodiments, the polymer nanoparticle delivers the nucleic acid to the brain or the cells of the central nervous system, for example microglia, astrocytes, oligodendrocytes, and neurons.

In one embodiment, a composition comprising a polymer nanoparticle (e.g., a polymer nanoparticle derived from a controlled living/radical polymerization process, such as RAFT polymer) associated with a nucleic acid (e.g., circRNA) construct is provided. In another embodiment, a method of treating a patient with a disease is provided comprising administering to the patient the polymer nanoparticle of the present disclosure. In another embodiment, a method of treating a patient with a disease is provided comprising administering to the patient the composition of the present disclosure.

In some embodiments, a method of treating a patient with a disease is provided, comprising administering to the patient the polymer nanoparticle identified in the in vivo screening method, wherein the polymer nanoparticle further comprises a drug payload, such as a polynucleotide or a protein payload, or a small molecule therapeutic or luminescent molecule payload, and treating the disease in the patient.

In various embodiments, any suitable route for administration of the library of polymer nanoparticles associated with nucleic acid constructs for the method of in vivo screening for the polymer nanoparticle associated with a nucleic acid construct, or for the method of treatment can be used including parenteral administration. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques. In other embodiments, oral or pulmonary routes of administration can be used.

In various embodiments, cell or tissue samples may be analyzed for the presence of the polymer nanoparticle associated with the nucleic acid constructs described herein. The samples can be any tissue, cell, or fluid sample from an animal, for example, selected from the group consisting of urine, nasal secretions, nasal washes, inner ear fluids, bronchial lavages, bronchial washes, alveolar lavages, spinal fluid, bone marrow aspirates, sputum, pleural fluids, synovial fluids, pericardial fluids, peritoneal fluids, saliva, tears, gastric secretions, stool, reproductive tract secretions, lymph fluid, whole blood, serum, plasma, or any tissue or cell sample from an animal. Exemplary tissue or cell samples include brain tissue or cells, muscle tissue or cells, skin tissue or cells, heart tissue or cells, kidney tissue or cells, stomach tissue or cells, liver tissue or cells, urinary tract tissue or cells, gastrointestinal tract tissue or cells, head or neck tissue or cells, lung tissue or cells, reproductive tract tissue or cells, pancreatic tissue or cells, or any other tissue or cell type from an animal.

In one illustrative aspect for removing cells or tissues from the animal and isolating the nucleic acid constructs from the cells or tissues of the animal, the nucleic acid constructs are removed from cells or tissues of the animal. In various embodiments, nucleic acid constructs (e.g., DNA or RNA) obtained from the tissues or cells of the animal can be removed by rupturing the cells and isolating the nucleic acid constructs from the lysate. Techniques for rupturing cells and for isolation of nucleic acids are well-known in the art, and removal techniques include homogenization, such as by using a bead-beating technique. In other embodiments, the nucleic acid constructs may be isolated by rupturing cells using a detergent or a solvent, such as phenol-chloroform. In another aspect, the nucleic acid constructs may be separated from the lysate by physical methods including, but not limited to, centrifugation, dialysis, diafiltration, filtration, size exclusion, pressure techniques, digestion of proteins with Proteinase K, or by using a substance with an affinity for nucleic acids such as, for example, beads that bind nucleic acids.

In various embodiments, payloads (e.g., nucleic acids) may be combined with the polymer nanoparticles compositions using any or all of covalent bonds, electrostatic interactions, and ligand affinity interactions. In one aspect, covalent bonding methods include the use of EDC/NHS to form stable amide bonds between the payload and the polymer nanoparticles for improved stability (both “on the shelf” and in vivo), ease of separation and extraction, and sensitive detection. In another illustrative aspect, electrostatic bonding methods include the use of cationic polymer nanoparticles that electrostatically complex with the payload. In another embodiment, ligand affinity bonding includes the use of ligands such as avidin and biotin, both covalently bonded to the polymer nanoparticles and the payload via EDC/NHS chemistry to yield the stable combination of the payload and the polymer nanoparticles.

It will be appreciated that RAFT polymerization is generally known in the art. Suitable reagents, monomers, and conditions for RAFT polymerization previously investigated can be used in the copolymers, methods, and compositions described herein, such as those described in U.S. Pat. Nos. 9,006,193, 9,464,300, and 9,476,063, the disclosures of each of which are incorporated by reference in their entirety.

Chain transfer agents (CTAs) useful in connection with the present disclosure are known in the art. The identity of the CTA is not particularly limited. It will be appreciated that chain transfers steps that form the basis of RAFT polymerization involve a reversible transfer of a functional chain end-group (typically a thiocarbonylthio group, Z—C(═S)S—R) between chains and the propagating radicals. The overall process is comprised of the insertion of monomers between the R- and Z—C(═S)S-groups of a RAFT agent (CTA), which form the α and ω end-group of the majority of the resulting polymeric chains. Suitable CTAs for use in connection with the present disclosure include but are not limited to trithiocarbonates (Z═S-alkyl), dithiobenzoates (Z=Ph), dithiocarbamate (Z═N-alkyl), xanthates (Z═O-alkyl), and the like. (See, S\tilde{e}bastien Perrier, Macromolecules 2017 50 (19), 7433-7447). In some embodiments, RAFT copolymerization may be achieved using chain transfer agents (CTAs) containing one or more terminal carboxyl groups in order to obtain carboxy terminated polymers with ends available for bonding to the payload via the methods described above. In this embodiment, when the resulting mono or di-carboxy terminated polymer is dispersed in a low pH (e.g., a pH of less than 6) buffer, both ends of the polymer are exposed and available for labeling via EDC/NHS chemistry. In this embodiment, when the polymer is transferred to a physiological pH (~pH 7), the core blocks self-assemble, encapsulating the payload in the hydrophobic core, to be released and exposed upon acidification in the endosomal compartment of a cell. In some embodiments, the first or second chain transfer agent can be selected from the group consisting of bis(carboxymethyl)trithiocarbonate, bis(2-amino-2-oxoethyl) trithiocarbonate, bis[4-(2-hydroxyethoxycarbonyl)benzyl]trithiocarbonate, 4-cyano-4-(ethylsulfanylthiocarbonyl) sulfanylvpentanoic acid, 4-cyano-4-((phenylcarbonothioyl)thio)pentanoic acid, and 4-cyano-4-[(dodecylsulfanylthiocarbonyl)sulfanyl]pentanoic acid, 4-cyano-4-(thiobenzoylthio)pentanoic acid, 2-cyano-2-propyl benzodithioate, cyanomethyl methyl(phenyl)carbamodithioate, 2-cyano-2-propyl dodecyl trithiocarbonate, 2-(dodecylthiocarbonothioylthio)-2-methylpropionic acid, cyanomethyl dodecyl trithiocarbonate, 2-cyano-2-propyl 4-cyanobenzodithioate, and the like.

In some embodiments, the block copolymer can be associated with a DNA molecule, in particular a nucleic acid construct of the present disclosure, via several methods including, electrostatic interaction, high affinity, non-covalent bond, avidin-streptavidin conjugation, or by direct covalent attachment through, for example, an amide bond. In some embodiments, the RAFT copolymer can be associated with a DNA molecule, in particular a nucleic acid construct of the present disclosure, via electrostatic interaction complexed with a biological molecule. In some embodiments, the block copolymer can be associated with a DNA molecule, in particular a nucleic acid construct of the present disclosure, via electrostatic interaction complexed with a biological molecule. In some embodiments, the block copolymer can be associated with a DNA molecule, in particular a nucleic acid construct of the present disclosure, via a high affinity, non-covalent bond, avidin-streptavidin conjugation. In some embodiments, the block copolymer can be associated with a DNA molecule, in particular a nucleic acid construct of the present disclosure, by direct covalent attachment through, for example, an amide bond.

The polymer nanoparticles described herein can be associated with a nucleic acid construct of the present disclosure via electrostatic interaction, avidin-streptavidin conjugation, or by direct covalent attachment. Exemplary interactions include: polymer nanoparticle (PNP) with positively charged corona in the case of electrostatic loading; nucleic acid constructs with negative charges due to the phosphate groups; electrostatically loaded PNP-nucleic acid construct complexes; carboxylate group on the terminal end of the polymer chains in the corona of the PNP; primary amine group on the 5′ end of the amine terminated nucleic acid construct; phosphate group on the 3′ end of the nucleic acid construct; amide bond formed in the direct amidification reaction between the amine terminal nucleic acid construct and the carboxylate terminated PNP; primary amine on the biotin bonding protein such as avidin; amide bond formed between the carboxylate group on the terminal end of the polymer chains in the corona of the PNP and the primary amine on the biotin bonding protein such as avidin; nucleic acid construct with a biotin functional group on the 5′ terminus; electrostatic coupling reaction that occurs when positively charged PNPs are mixed with negatively charged nucleic acid constructs; direct amidification reaction that is carried out via an EDA-NHC reaction between the carboxylate group on the terminal end of the polymer chains in the corona of the PNP and the primary amine on the amine terminated nucleic acid constructs; direct amidification reaction that is carried out via an EDA-NHC reaction between the carboxylate group on the terminal end of the polymer chains in the corona of the PNP and the primary amine on the biotin bonding protein such as avidin; coupling of the biotin on the 5′ end of the nucleic acid construct and the avidin conjugated to the carboxylate terminus on the corona of the PNPs.

In another illustrative embodiment, the polymer nanoparticle composition can be coated with one or more polymers to protect the compositions from immune responses or to enhance endosomal escape. In one embodiment, the one or more polymers used for coating comprise polyethylene glycol. In another embodiment, the one or more polymers used for coating comprise polyethylene glycol poly-L-lysine. In yet another embodiment, the one or more polymers used for coating comprise polyethylenimine. In an additional embodiment, the one or more polymers used for coating comprise polyethylene glycol poly-L-lysine and polyethylenimine.

It will be appreciated that tuning the parameters and properties of the block copolymers described herein can be advantageous to their use in the compositions and methods as described herein. Accordingly, the methods for preparing block copolymers either in singleton or in library format as described herein are capable of providing particular parameters and properties of the block copolymers.

In some embodiments, a single chain transfer agent can be used in the RAFT polymerization process in connection with the present disclosure. In some embodiments, for a block polymer having more than one block, one or more single chain transfer agents can be used in the RAFT polymerization process in connection with the present disclosure. In some embodiments, for a block polymer having two blocks, a first chain transfer agent and a second chain transfer agent (which can be the same or different) can be used at each step of the RAFT polymerization process in connection with the present disclosure. In some embodiments, for a block polymer having three blocks, a first chain transfer agent, a second chain transfer agent, and a third chain transfer agent (which can be the same or different) can be used at each step of the RAFT polymerization process in connection with the present disclosure.

It will be appreciated that a variety of solvents can be used in the RAFT polymerization method steps and purification steps described herein. Suitable solvents include, but are not limited to, 2-Chloroethanol, Acetic Acid (Glacial), Acetone, Acetonitrile, Acetophenone, Aniline, Benzaldehyde, Benzyl Acetate, Carbon disulfide, Cyclohexane, Cyclohexanol, Di(ethylene glycol), Di(propylene glycol), Diacetone alcohol, Diethyl ether, Dimethylsulfoxide, Ethanol, Ethyl acetate, Ethylene glycol, Formaldehyde (37% solution), Formamide, Formic acid, Formic acid (96%), HexaneIsobutanol, Isopropanol, Isopropyl acetate, Isopropyl ether, m-Cresol, Methanol, Methyl acetate, Methyl ethyl ketone, Mineral Oil, N,N-Dimethylformamide, n-Butanol, n-Octane, n-Propanol, Propylene glycol, Pyridine, t-Butanol, Tetrahydrofuran, Trifluoroacetic acid, water, and the like, and combinations thereof.

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exists a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

### Alternative Embodiments

The following clauses, and combinations thereof, provide various additional illustrative aspects of the disclosure described herein. The various embodiments described in any other section of this patent application, including the section titled “DETAILED DESCRIPTION,” “REPRESENTATIVE EMBODIMENTS,” and the “EXAMPLES” are applicable to any of the following embodiments described in the clauses enumerated below.

1. A block copolymer comprising
a first block comprising a monomer unit represented by formula I:

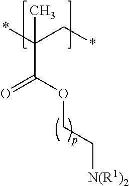

or a salt thereof, wherein:
   p is 0 or an integer selected from 1-3,
   each R¹ is individually selected from H or alkyl; and
   wherein each * represents a connecting point to the rest of the block copolymer; and
a second block comprising a copolymer of monomer units selected from formula II:

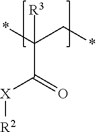

or a salt thereof, wherein:
   X is —O—;
   R² is alkyl; and
   R³ is alkyl;
   wherein each * represents a connecting point to the rest of the block copolymer; and
   formula III:

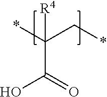

or a salt thereof, wherein:
   R⁴ is alkyl;
   wherein each * represents a connecting point to the rest of the block copolymer.
      2. The block copolymer of clause 1, wherein p is 1.
      3. The block copolymer of clause 1 or 2, wherein each R¹ is independently C₁-C₃ alkyl.
      4. The block copolymer of clause 1 or 2, wherein each R¹ is methyl.
      5. The block copolymer of any one of clauses 1-4, wherein R² is C₁-C₆ alkyl.
      6. The block copolymer of any one of clauses 1-5, wherein R² is butyl.
      7. The block copolymer of any one of clauses 1-6, wherein R³ is C₁-C₆ alkyl.
      8. The block copolymer of any one of clauses 1-7, wherein R³ is methyl.
      9. The block copolymer of any one of clauses 1-8, wherein R⁴ is C₁-C₆ alkyl.
      10. The block copolymer of any one of clauses 1-9, wherein R⁴ is ethyl or propyl.
      11. The block copolymer of any one of clauses 1-10, wherein R⁴ is ethyl.
      12. The block copolymer of any one of clauses 1-10, wherein R⁴ is propyl.
      13. A block copolymer comprising a first block and a second block, wherein:
the first block comprises a homopolymer of poly(2-dimethylaminoethyl methacrylate) p(DMAEMA), and
the second block comprises a copolymer of an alkylacrylate (e.g., butylmethacrylate (BMA)) and an acrylic acid (e.g., methacrylic acid (MAA), ethylacrylic acid (EAA) or propylacrylic acid (PAA)).
   14. The block copolymer of any one of the preceding clauses, selected from p(DMAEMA)-b-p(BMA-co-MAA), p(DMAEMA)-b-p(BMA-co-EAA), and p(DMAEMA)-b-p(BMA-co-PAA).
   15. The block copolymer of any one of the preceding clauses, wherein the copolymer has a total molecular weight of about 20 kDa to about 65 kDa.
   16. The block copolymer of any one of the preceding clauses, wherein the copolymer has a total molecular weight of about 25 kDa to about 60 kDa.
   17. The block copolymer of any one of the preceding clauses, wherein the first block has a molecular weight of about 15,000 Da to about 55,000 Da.
   18. The block copolymer of any one of the preceding clauses, wherein the first block has a molecular weight of about 20,000 Da to about 55,000 Da.
   19. The block copolymer of any one of the preceding clauses, wherein the second block has a degree of polymerization of about 15 to about 175.
   20. The block copolymer of any one of the preceding clauses, wherein the second block has a degree of polymerization of about 40 to about 80.
   21. The block copolymer of any one of the preceding clauses, wherein the ratio of the molecular weights of the compound of formula II and the compound of formula III in the second block is about 30:70 to about 70:30.
   22. The block copolymer of any one of the preceding clauses, wherein the first block comprises a cap of formula:

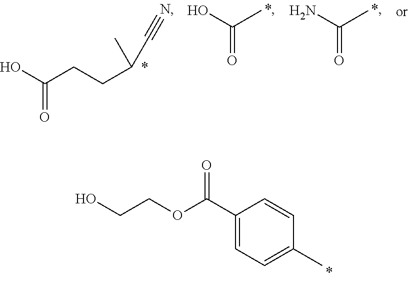

or a salt thereof, wherein * represents a point of covalent attachment to the first block.
   23. The block copolymer of any one of the preceding clauses, wherein the first block comprises a cap of formula:

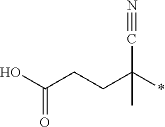 [SMILES: *C(C)(C#N)CCC(=O)O]

or a salt thereof, wherein the * represents a point of connection to the first block.
   24. The block copolymer of any one of the preceding clauses, wherein the second block comprises a cap of formula:

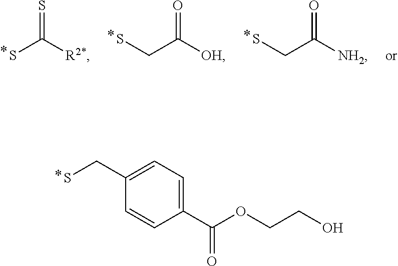

or a salt thereof, wherein * represents a point of covalent attachment to the second block, and R²* is —SC₂-C₁₂ alkyl or C₆H₅.
   25. The block copolymer of any one of the preceding clauses, wherein the second block comprises a cap of formula:

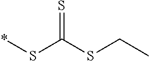 [SMILES: *SC(=S)SCC]

or a salt thereof, wherein the * represents a point of connection to the second block.
   26. A polymer nanoparticle comprising:
the block copolymer according to any one of clauses 1-25.
   27. A composition comprising:
a polymer nanoparticle according to clause 26, and
a circular RNA complexed to the polymer nanoparticle,
wherein the ratio by weight of the nanoparticle to the circular RNA is about 1:1 to about 25:1.
   28. The composition of clause 27, wherein the polymer nanoparticle is complexed to the circular RNA via electrostatic interaction.
   29. The composition of clause 27 or 28, wherein the ratio by weight of the nanoparticle to the circular RNA is about 1:1 to about 20:1.
   30. The composition of clause 27 or 28, wherein the ratio by weight of the nanoparticle to the circular RNA is about 1:1 to about 10:1.
   31. The composition of clause 27 or 28, wherein the ratio by weight of the nanoparticle to the circular RNA is about 1:1 to about 7:1.
   32. The composition of clause 27 or 28, wherein the ratio by weight of the nanoparticle to the circular RNA is about 1:1 to about 5:1.
   33. The composition of any one of clauses 27-32, wherein the transfection efficiency to a cell is at least about 90%.
   34. The composition of any one of clauses 27-32, wherein the transfection efficiency to a cell is at least about 95%.
   35. The composition of clause 33 or 34, wherein the cell is a mammalian cell (e.g., a human cell).
   36. The composition of any one of clauses 27-35, further comprising a pharmaceutically acceptable carrier.
   37. A method of treating a disease in a patient in need thereof, the method comprising:
administering a therapeutically effective amount of a composition according to any one of clauses 27-35.
   38. A method of transfecting a cell, the method comprising:
contacting the cell with an effective amount of a composition according to any one of clauses 27-35.
   39. The method of clause 38, wherein the method is in vitro.
   40. The method of clause 38, wherein the method is in vivo.

### Additional Alternative Embodiments

The following embodiments, and combinations thereof, provide various additional illustrative aspects of the disclosure described herein. The various embodiments described in any other section of this patent application, including the section titled “DETAILED DESCRIPTION,” “REPRESENTATIVE EMBODIMENTS,” and the “EXAMPLES” are applicable to any of the following embodiments described in the embodiments enumerated below.

1. A composition comprising:
a polymer nanoparticle comprising a block copolymer, and
a circular RNA complexed to the polymer nanoparticle, wherein the ratio by weight of the nanoparticle to the circular RNA is about 1:1 to about 20:1, and
wherein the block copolymer comprises:
a first block comprising a monomer unit represented by formula I:

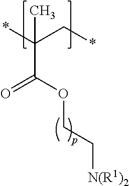

or a salt thereof, wherein:
   p is 0 or an integer selected from 1-3,
   each R¹ is individually selected from H or alkyl; and
   wherein each * represents a connecting point to the rest of the block copolymer; and
a second block comprising a copolymer of monomer units selected from formula II:

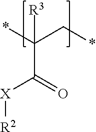

or a salt thereof, wherein:
   X is —O—;
   R² is alkyl; and
   R³ is alkyl;
   wherein each * represents a connecting point to the rest of the block copolymer; and
   formula III:

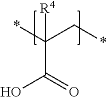

or a salt thereof, wherein:
   R⁴ is alkyl;
   wherein each * represents a connecting point to the rest of the block copolymer.
      2. The composition of embodiment 1, wherein p is 1.
      3. The composition of embodiment 1 or 2, wherein each R¹ is independently C₁-C₃ alkyl.
      4. The composition of embodiment 1 or 2, wherein each R¹ is methyl.
      5. The composition of any one of embodiments 1-4, wherein R² is C₁-C₆ alkyl.
      6. The composition of any one of embodiments 1-5, wherein R² is butyl.
      7. The composition of any one of embodiments 1-6, wherein R³ is C₁-C₆ alkyl.
      8. The composition of any one of embodiments 1-7, wherein R³ is methyl.
      9. The composition of any one of embodiments 1-8, wherein R⁴ is C₁-C₆ alkyl.
      10. The composition of any one of embodiments 1-9, wherein R⁴ is ethyl or propyl.
      11. The composition of any one of embodiments 1-10, wherein R⁴ is ethyl.
      12. The composition of any one of embodiments 1-10, wherein R⁴ is propyl.
      13. A composition comprising:
a polymer nanoparticle comprising a block copolymer, and
a circular RNA complexed to the polymer nanoparticle, wherein the ratio by weight of the nanoparticle to the circular RNA is about 1:1 to about 20:1, and
wherein the block copolymer comprises:
a first block comprising a homopolymer of poly(2-dimethylaminoethyl methacrylate) (p(DMAEMA)), and
a second block comprising a copolymer of an alkylacrylate (e.g., butylmethacrylate (BMA)) and an acrylic acid (e.g., methacrylic acid (MAA), ethylacrylic acid (EAA) or propylacrylic acid (PAA)).
   14. The composition of any one of the preceding embodiments, wherein the block copolymer is selected from the group consisting of p(DMAEMA)-b-p(BMA-co-MAA), p(DMAEMA)-b-p(BMA-co-EAA), and p(DMAEMA)-b-p(BMA-co-PAA).
   15. The composition of any one of the preceding embodiments, wherein the copolymer has a total molecular weight of about 20 kDa to about 65 kDa.
   16. The composition of any one of the preceding embodiments, wherein the copolymer has a total molecular weight of about 25 kDa to about 60 kDa.
   17. The composition of any one of the preceding embodiments, wherein the first block has a molecular weight of about 15,000 Da to about 55,000 Da.
   18. The composition of any one of the preceding embodiments, wherein the first block has a molecular weight of about 20,000 Da to about 55,000 Da.
   19. The composition of any one of the preceding embodiments, wherein the second block has a degree of polymerization of about 15 to about 175.
   20. The composition of any one of the preceding embodiments, wherein the second block has a degree of polymerization of about 40 to about 80.
   21. The composition of any one of the preceding embodiments, wherein the ratio of the molecular weights of the compound of formula II and the compound of formula III in the second block is about 30:70 to about 70:30.
   22. The composition of any one of the preceding embodiments, wherein the ratio of the molecular weights of the compound of formula II and the compound of formula III in the second block is about 40:60 to about 60:40.
   23. The composition of any one of the preceding embodiments, wherein the first block comprises a cap of formula:

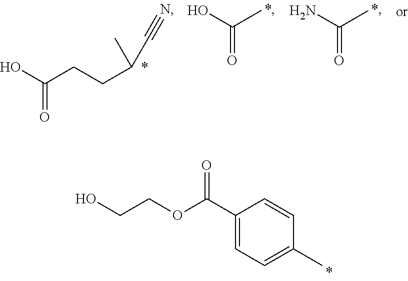

or a salt thereof, wherein * represents a point of covalent attachment to the first block.
   24. The composition of any one of the preceding embodiments, wherein the first block comprises a cap of formula:

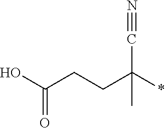 [SMILES: *C(C)(C#N)CCC(=O)O]

or a salt thereof, wherein the * represents a point of connection to the first block.
   25. The composition of any one of the preceding embodiments, wherein the second block comprises a cap of formula:

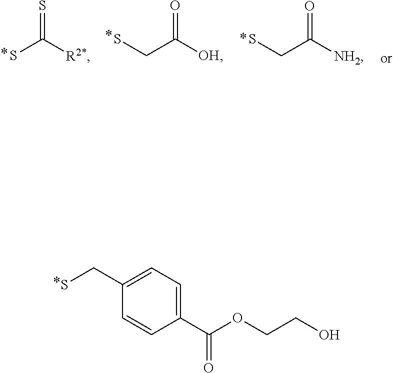

or a salt thereof, wherein * represents a point of covalent attachment to the second block, and R²* is —SC₂-C₁₂ alkyl or C₆H₅.
   26. The block copolymer of any one of the preceding embodiments, wherein the second block comprises a cap of formula:

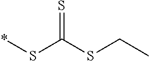 [SMILES: *SC(=S)SCC]

or a salt thereof, wherein the * represents a point of connection to the second block.
   27. The composition of any one of the preceding embodiments, wherein the polymer nanoparticle is complexed to the circular RNA via electrostatic interaction.
   28. The composition of any one of the preceding embodiments, wherein the ratio by weight of the nanoparticle to the circular RNA is about 1:1 to about 10:1.
   29. The composition of any one of the preceding embodiments, wherein the ratio by weight of the nanoparticle to the circular RNA is about 1:1 to about 7:1.
   30. The composition of any one of the preceding embodiments, wherein the ratio by weight of the nanoparticle to the circular RNA is about 1:1 to about 5:1.
   31. The composition of any one of any one of the preceding embodiments, wherein the transfection efficiency to a cell is at least about 90%.
   32. The composition of any one of any one of the preceding embodiments, wherein the transfection efficiency to a cell is at least about 95%.
   33. The composition of embodiment 31 or 32, wherein the cell is a mammalian cell (e.g., a human cell).
   34. The composition of any one of the preceding embodiments, further comprising a pharmaceutically acceptable carrier.
   35. A method of treating a disease in a patient in need thereof, the method comprising: administering a therapeutically effective amount of a composition according to any one of any one of the preceding embodiments.
   36. A method of transfecting a cell, the method comprising:
contacting the cell with an effective amount of a composition according to any one of embodiments 1-35.
   37. The method of embodiment 36, wherein the method is in vitro.
   38. The method of embodiment 36, wherein the method is in vivo.

### Examples

### Polymer Nanoparticle Synthesis

Diblock copolymers were synthesized similarly as described in U.S. Patent Application Publication No. 2022/0175812, the entirety of which is incorporated by reference herein, with some modifications using reversible addition-fragmentation chain transfer (RAFT) polymerization with reagents and amounts listed in Table 2. Block 1 reagents, including 2-(N,N-Dimethylamino)ethyl methacrylate (DMAEMA) as the monomer, 4-Cyano-4-[(ethylsulfanylthiocarbonyl)sulfanyl]pentanoic acid (ECT) as the chain transfer agent (CTA), 2,2′-Azobis(2-methylpropionitrile (AIBN) as the initiator, and N,N-Dimethylformamide (DMF) as the solvent, were combined in a 100 mL round-bottom flasks, purged with argon, and heated to 70° C. for 24 hours using either an oil bath. The reaction products were purified using four 80:20 pentane:ether precipitation washes and centrifugation cycles and dried in vacuo. The Block 1 products were characterized for molecular weight using static light scattering (SLS) by a DynaPro Plate Reader III and/or by right-angle light scattering (RALS) coupled with low-angle light scattering (LALS) plus refractive index, viscosity, and concentration detection by the Malvern OMNISEC GPC. The Block 1 products were used as the macroRAFT agents for Block 2, and the calculated reagent volumes (as calculated based on the SLS/LALS/RALS theoretical molecular weight information for Block 1) were combined with AIBN initiator, butyl methacrylate monomer (BMA), and propyl acrylic acid (PAA) monomers in 1 mL glass shell vials for the Block 2 reactions. The reaction mixtures were purged with argon before being heated at 70° C. for 24 hours. The reaction products were purified using four 80:20 pentane:ether precipitation washes and centrifugation cycles followed by drying in vacuo. Finally, the purified materials were lyophilized for ~3-4 days and stored at room temperature for experimental use.

Block copolymers were prepared using Reversible Addition-Fragmentation Chain Transfer (RAFT) polymerization, using the monomers shown in Table 1 below. The RAFT polymerization process produced the following block copolymers:

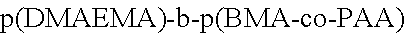

with Block 1 theoretical degree of polymerization values ranging from 360 to 600 (measured Block 1 MW range from ~10 kDa to ~50 kDa), and block 2 theoretical degree of polymerization values ranging from 50 to 85, resulting in actual overall diblock copolymer molecular weight range from ~13 kDa to ~50 kDa.

TABLE 1
Monomers used in the nanoparticle forming polymers (PNPs).

| Monomer Name | Abbreviation | CAS | Structure |
|---|---|---|---|
| Propylacrylic acid | PAA | 5650-75-9 | 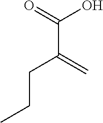 |
| Butyl methacrylate | BMA | 97-88-1 | 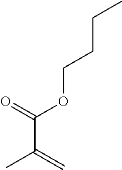 |
| Dimethylaminoethyl methacrylate | DMAEMA | 2867-47-2 | 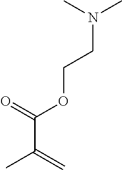 |

TABLE 2
PNP composition summary

| | B1 | B2 | mCTA | | % | | % |
|---|---|---|---|---|---|---|---|
| | DMAEMA | BMA-co-PAA | Mw | Monomer | Monomer 1 | Monomer | Monomer 2 |
| PNP | Theoretical DP | Theoretical DP | (kDa) | 1 | in B2 | 2 | in B2 |
| 1 | 360 | 50 | 19.1 | BMA | 51.3 | PAA | 48.7 |
| 2 | 360 | 60 | 19.1 | BMA | 56.2 | PAA | 43.8 |
| 3 | 360 | 70 | 19.1 | BMA | 66.0 | PAA | 34.0 |
| 4 | 480 | 50 | 36.3 | BMA | 44.4 | PAA | 55.6 |
| 5 | 480 | 60 | 36.3 | BMA | 46.8 | PAA | 53.2 |
| 6 | 480 | 70 | 36.3 | BMA | 50.0 | PAA | 50.0 |
| 7 | 600 | 50 | 45.5 | BMA | 43.3 | PAA | 56.7 |
| 8 | 600 | 78 | 45.5 | BMA | 50.5 | PAA | 49.5 |
| 9 | 600 | 60 | 45.5 | BMA | 49.6 | PAA | 50.4 |
| 10 | 600 | 70 | 45.5 | BMA | 44.1 | PAA | 55.9 |

### Transfection Efficiency and Viability in H1EK293T Cells

To measure the in vitro transfection efficiency and viability upon transfection, the PNPs were loaded with a circular RNA (circRNA) expressing the fluorescent eGFP reporter protein. These were dosed into HTEK293T cells.

For PNP IDs 1-10, cells were seeded in 96 well plates at 30,000 cells per well and incubated for 24 hours before treating with 6 different doses of PNP and circRNA cargo:

| mg/mL PNP | ng circRNA | PNP:circRNA ratio |
|---|---|---|
| 0.0075 | 150 | 5 |
| 0.015 | 150 | 10 |
| 0.03 | 150 | 20 |
| 0.0075 | 300 | 2.5 |
| 0.015 | 300 | 5 |
| 0.03 | 300 | 10 |

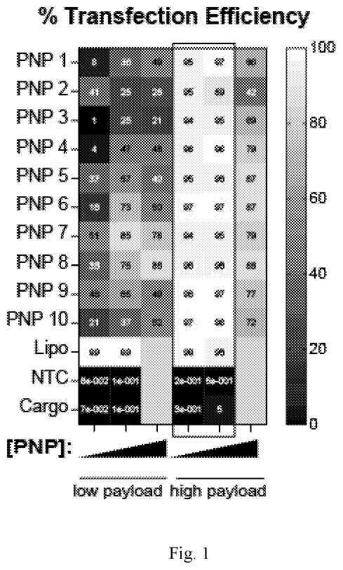

The number of GFP-expressing cells were counted via flow cytometry at 48 hours post-dosing. Transfection efficiency was calculated as defined by the equation below. Results are shown for PNP Is 1-10 in Table 3 and FIG. 1.

$$Transfection efficiency ( \% ) = \frac{GFP expressing Cells}{Total Cells}$$

TABLE 3
Transfection Efficiency upon eGFP circRNA transfection

| | 0.0075 | 0.015 | 0.03 | 0.0075 | 0.015 | 0.03 |
|---|---|---|---|---|---|---|
| | mg/ml PNP, | mg/ml PNP, | mg/ml PNP, | mg/ml PNP, | mg/ml PNP, | mg/ml PNP, |
| | 150 ng | 150 ng | 150 ng | 300 ng | 300 ng | 300 ng |
| PNP | circRNA | circRNA | circRNA | circRNA | circRNA | circRNA |
| ID | (%) | (%) | (%) | (%) | (%) | (%) |
| 1 | 8.4 | 36.0 | 49.2 | 94.9 | 96.6 | 68.3 |
| 2 | 41.1 | 24.7 | 26.1 | 94.9 | 89.0 | 42.3 |
| 3 | 0.6 | 25.2 | 21.4 | 94.4 | 95.0 | 69.5 |
| 4 | 4.0 | 46.5 | 47.7 | 96.0 | 97.6 | 79.3 |
| 5 | 37.4 | 56.6 | 40.1 | 95.3 | 96.2 | 87.2 |
| 6 | 15.9 | 73.0 | 49.7 | 96.7 | 96.5 | 87.0 |
| 7 | 51.1 | 85.0 | 77.9 | 93.9 | 94.9 | 79.2 |
| 8 | 35.5 | 74.8 | 86.4 | 96.1 | 96.4 | 86.3 |
| 9 | 48.4 | 65.2 | 48.6 | 97.7 | 97.5 | 77.3 |
| 10 | 21.5 | 36.8 | 51.6 | 96.9 | 98.3 | 71.8 |

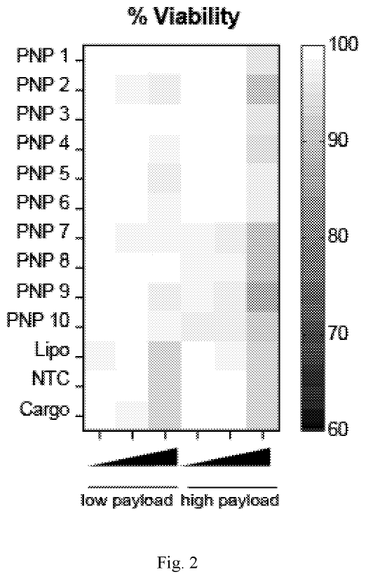

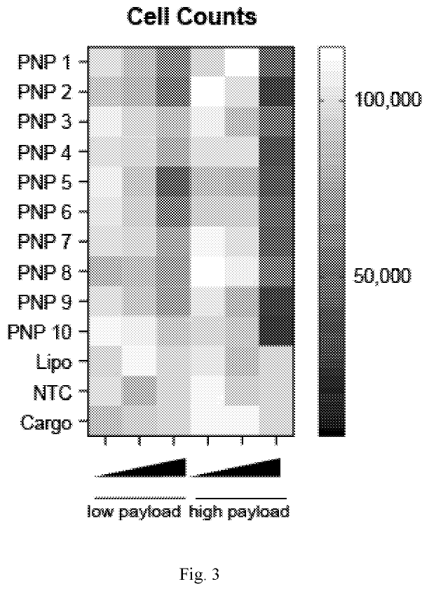

Viability data was also captured by staining cells with zombie violet, which specifically only stains dead cells. Viability 00 was calculated as defined by the equation below. Results are shown for PNP IDs 1-10 in Table 4 and FIGS. 2-3.

$$Cell Viability ( \% ) = \frac{non - Violet cells}{Total Cells}$$

TABLE 4
Cell Viability upon eGFP circRNA transfection

| | 0.0075 | 0.015 | 0.03 | 0.0075 | 0.015 | 0.03 |
|---|---|---|---|---|---|---|
| | mg/ml PNP, | mg/ml PNP, | mg/ml PNP, | mg/ml PNP, | mg/ml PNP, | mg/ml PNP, |
| PNP | 150 ng | 150 ng | 150 ng | 300 ng | 300 ng | 300 ng |
| ID | circRNA | circRNA | circRNA | circRNA | circRNA | circRNA |
| 1 | 99.5 | 99.1 | 98.6 | 99.7 | 99.4 | 95.9 |
| 2 | 99.2 | 98.3 | 97.4 | 99.8 | 99.4 | 90.9 |
| 3 | 99.5 | 99.0 | 99.2 | 99.7 | 99.4 | 95.8 |
| 4 | 99.3 | 98.8 | 98.4 | 99.5 | 99.5 | 94.9 |
| 5 | 99.4 | 98.9 | 96.1 | 99.7 | 99.3 | 96.1 |
| 6 | 99.4 | 98.8 | 97.9 | 99.6 | 99.3 | 96.5 |
| 7 | 98.8 | 98.5 | 97.6 | 98.1 | 97.3 | 90.9 |
| 8 | 99.1 | 98.7 | 98.7 | 98.4 | 97.6 | 91.1 |
| 9 | 99.0 | 98.8 | 97.3 | 97.7 | 96.4 | 86.8 |
| 10 | 99.1 | 98.9 | 98.3 | 97.4 | 96.9 | 92.6 |

1. A composition comprising:
a polymer nanoparticle comprising a block copolymer, and
a circular RNA complexed to the polymer nanoparticle, wherein the ratio by weight of the nanoparticle to the circular RNA is about 1:1 to about 20:1, and
wherein the block copolymer comprises:
a first block comprising a monomer unit represented by formula I: 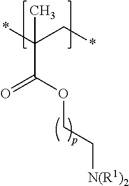
or a salt thereof, wherein:
p is 0 or an integer selected from 1-3,
each R¹ is individually selected from H or alkyl; and
wherein each * represents a connecting point to the rest of the block copolymer; and
a second block comprising a copolymer of monomer units selected from formula II: 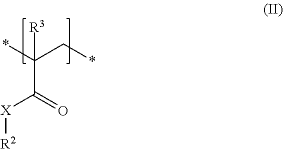
or a salt thereof, wherein:
X is —O—;
R² is alkyl; and
R³ is alkyl;
wherein each * represents a connecting point to the rest of the block copolymer; and
formula III: 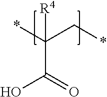
or a salt thereof, wherein:
R⁴ is alkyl;
wherein each * represents a connecting point to the rest of the block copolymer.

2. The composition of claim 1, wherein p is 1.

3. The composition of claim 1, wherein each R¹ is independently C₁-C₃ alkyl.

4. The composition of claim 1, wherein each of R², R³, and R⁴ is independently C₁-C₆ alkyl.

5. The composition of claim 1, wherein the first block comprises a homopolymer of poly(2-dimethylaminoethyl methacrylate) (p(DMAEMA)).

6. The composition of claim 5, wherein the block copolymer is selected from the group consisting of p(DMAEMA)-b-p(BMA-co-MAA), p(DMAEMA)-b-p(BMA-co-EAA), and p(DMAEMA)-b-p(BMA-co-PAA).

7. The composition of claim 1, wherein the copolymer has a total molecular weight of about 20 kDa to about 65 kDa.

8. The composition of claim 1, wherein the first block has a molecular weight of about 15,000 Da to about 55,000 Da.

9. The composition of claim 1, wherein the second block has a degree of polymerization of about 40 to about 80.

10. The composition of claim 1, wherein the ratio of the molecular weights of the compound of formula II and the compound of formula III in the second block is about 30:70 to about 70:30.

11. The composition of claim 1, wherein the first block comprises a cap of formula: 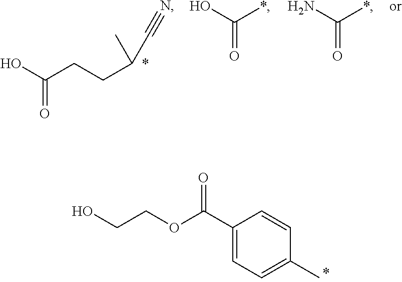
or a salt thereof, wherein * represents a point of covalent attachment to the first block.

12. The block copolymer of claim 1, wherein the second block comprises a cap of formula: 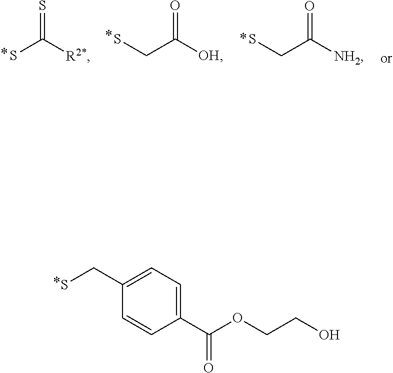
or a salt thereof, wherein * represents a point of covalent attachment to the second block, and R²* is —SC₂-C₁₂ alkyl or C₆H₅.

13. The composition of claim 1, wherein the polymer nanoparticle is complexed to the circular RNA via electrostatic interaction.

14. The composition of claim 1, wherein the ratio by weight of the nanoparticle to the circular RNA is about 1:1 to about 10:1.

15. The composition of claim 14, wherein the ratio by weight of the nanoparticle to the circular RNA is about 1:1 to about 7:1.

16. The composition of claim 1, wherein the transfection efficiency to a cell is at least about 90%.

17. The composition of claim 16, wherein the cell is a mammalian cell.

18. The composition of claim 1, further comprising a pharmaceutically acceptable carrier.

19. A method of treating a disease in a patient in need thereof, the method comprising:
administering a therapeutically effective amount of a composition according to claim 1.

20. A method of transfecting a cell, the method comprising:
contacting the cell with an effective amount of a composition according to claim 1.
